(19) United States
(12) Patent Application Publication
(10) Pub. No.: US 2004/0175829 A1
(43) Pub. Date: Sep. 9, 2004

(54) Nucleocapsid-independent specific viral RNA packaging and uses thereof

(76) Inventors: Shinji Makino, Galveston, TX (US); Krishna Narayanan, Galveston, TX (US)

Correspondence Address: Benjamin Aaron Adler, ADLER & ASSOCIATES, 8011 Candle Lane, Houston, TX, 77071, US

(21) Appl. No.: 10/794,363

(22) Filed: Mar. 5, 2004

Related U.S. Application Data
(60) Provisional application No. 60/452,402, filed on Mar. 6, 2003.

(51) Int. Cl. C12N 15/86; C12N 7/00

(52) U.S. Cl. 435/456; 435/235.1

(57) ABSTRACT

The present invention shows that expressed coronavirus envelope protein M specifically interacted with co-expressed non-coronavirus RNA transcripts containing the short viral packaging signal in the absence of coronavirus N protein. Furthermore, this M protein-packaging signal interaction led to specific packaging of the packaging-signal-containing RNA transcripts into coronavirus-like particles in the absence of N protein. These findings highlight a novel RNA packaging mechanism for an enveloped virus, and a novel coronavirus-based expression system can be developed based on the data presented herein.

### CROSS REFERENCE TO RELATED APPLICATION

This non-provisional application claims benefit of provisional application U.S. Serial No. 60/452,402, filed on Mar. 6, 2003 and now abandoned.

### FEDERAL FUNDING LEGEND

This invention was produced in part using funds obtained through a grant A129984 from the National Institutes of Health. Consequently, the federal government has certain rights in this invention.

### BACKGROUND OF THE INVENTION

1. Field of the Invention

The present invention relates generally to the field of viral assembly. More specifically, it relates to nucleocapsid-independent viral RNA packaging and uses thereof.

2. Description of the Related Art

At a certain moment in the life cycle of viruses, when sufficient copies of the viral genome have been synthesized, the genomic RNA has to be incorporated into a new virus particle to form progeny virus. This virus particle has the important task to transport the genome to another susceptible host and to provide a stable protective environment for the genome.

For non-enveloped viruses, the encapsidation of the genomic RNA into a functional ribonucleoprotein particle is sufficient to form infectious virions. For enveloped viruses, the formation of an infectious particle requires the interaction of the ribonucleoprotein complex (or nucleocapsid) with the viral envelope glycoproteins and/or the phospholipid membrane, followed by the budding of this nucleocapsid through cellular membranes. It is clear that the process of RNA encapsidation into ribonucleoprotein complexes is of great importance for the replication of viruses, either enveloped or non-enveloped.

During assembly, the virus should package only its own genome. Obviously, a highly specific mechanism must be responsible for the selection of genomic RNA from the pool of cellular mRNAs, rRNAs, tRNAs, viral subgenomic RNAs and intermediates of the opposite polarity. For a large number of viruses, a specific RNA signal has been identified that directs the encapsidation of the viral genomic RNA.

For enveloped RNA viruses, the association of an intracellular form of viral genomic RNA with the nucleocapsid/capsid protein is the first step in the process of selective genome packaging into virus particles. A specific RNA element(s), usually referred to as a packaging signal or an encapsidation signal, that is present in intracellular viral genomic RNA determines the selective and specific binding of viral nucleocapsid protein to the viral genomic RNA. Subsequently, the viral ribonucleoprotein (RNP) complex containing viral RNA and the nucleocapsid protein binds to a viral envelope protein(s) at the virus budding site, which leads to the budding of virus particles containing the viral ribonucleoprotein complex.

In some enveloped viruses, an interaction between the viral ribonucleoprotein complex and envelope proteins drives the budding of virus particles; while in other enveloped viruses, the viral ribonucleoprotein complex is dispensable for viral envelope formation and production of virus particles. A typical example of the latter phenomenon is observed in coronavirus envelope formation. Coronavirus-like particles (VLPs) that are morphologically similar to infectious virus particles are produced in the absence of viral ribonucleoprotein complex (Vennema et al., 1996).

Coronavirus Family

The coronavirus family comprises twelve species divided into three serological groups (I, II and III) (reviewed in Lai and Cavanagh, 1997). Group I coronaviruses include transmissible gastroenteritis virus, feline coronavirus, canine coronavirus, human coronavirus 229E and porcine epidemic diarrhea virus. Group II coronaviruses include mouse hepatitis virus, bovine coronavirus, human coronavirus OC43, porcine hemagglutinating encephalomyelitis virus and sialodacryoadenitis virus. Group III coronaviruses include turkey coronavirus and infectious bronchitis virus.

The murine coronavirus, mouse hepatitis virus (MHV) contains three envelope proteins, S protein, M protein and E protein and a helical nucleocapsid consisting of N protein and a large single-stranded, positive-stranded RNA genome (Lai and Stohlman, 1978; Sturman et al., 1980). S protein is dispensable for viral nucleocapsid packaging and viral assembly. M protein and E protein are essential for viral envelope formation and release of virus particles. Coronavirus-like particles are released from cells that express both M protein and E protein (Vennema et al., 1996).

The M protein, the most abundant transmembrane envelope glycoprotein in the virus particle and in infected cells, is characterized as having three domains: a short N-terminal ectodomain, a triple-spanning transmembrane domain and a C-terminal endodomain (Armstrong et al., 1984). E protein is present only in minute amounts in infected cells and in the viral envelope, yet it plays a central role in coronavirus morphogenesis (Fischer et al., 1998). The viral genomic RNA and N protein form the helical nucleocapsid structure, which exists, inside the viral envelope (Escors et al., 2001; Sturman et al., 1980).

In infected cells, mouse hepatitis virus synthesizes the intracellular form of genomic RNA, mRNA 1, and six to seven species of subgenomic mRNAs. These virus-specific mRNAs comprise a nested set with a common 3′ terminus and a common leader sequence of approximately 72 to 77 nucleotides (nt) at the 5′ end (Lai et al., 1984; Spaan et al., 1983). All mouse hepatitis virus mRNAs associate with N protein to form ribonucleoprotein complexes (Baric et al., 1988; Narayanan et al., 2000); however, only the mRNA 1-ribonucleoprotein complex is efficiently packaged into the virus particles.

Previous studies demonstrated that only mRNA 1 and the viral genomic RNA, but not subgenomic mRNAs, contain a 190 nt-long packaging signal (PS) (Fosmire et al., 1992; van der Most et al., 1991). A specific interaction occurs between the viral transmembrane envelope protein M and mRNA 1-N protein complex at the budding site in infected cells (Narayanan et al., 2000), and the 190 nt-long packaging signal mediates the specific interaction between M protein and mRNA 1-N protein complex (or other ribonucleoprotein complexes containing the packaging signal) to drive the specific packaging of RNA into virus particles (Narayanan and Makino, 2001). How M protein selectively and specifically recognizes the packaging signal-containing ribonucleoprotein complex is unknown.

Two models have been suggested to explain the mechanism of specific recognition of packaging signal-containing ribonucleoprotein complexes by M protein (Narayanan and Makino, 2001). One was that M protein recognizes a specific helical nucleocapsid structure formed by the mRNA 1-N protein complex. The binding of N protein to the packaging signal might trigger the formation of helical nucleocapsid structure. In this model, both M protein and N protein contribute to the selective packaging of specific RNA species into virus particles. Another model was that the direct interaction of M protein with the packaging signal in the packaging signal-containing ribonucleoprotein complex is responsible for the selectivity in RNA packaging.

The present invention presents data that support the second model described above. In contrast to other enveloped RNA viruses in which recognition of a specific RNA packaging signal by the virus's nucleocapsid (N) protein is the first step in the process of viral RNA packaging, the present invention describes an interaction between M protein and packaging signal that led to specific packaging of the packaging-signal-containing RNA transcripts into coronavirus-like particles in the absence of N protein. These findings not only highlight a novel RNA packaging mechanism for an enveloped virus, but also point to a new, biologically important general model of precise and selective interaction between transmembrane proteins and specific RNA elements.

The prior art is deficient in a coronavirus-based expression vector system for delivery of RNA and expression of proteins in eukaryotic cells. The present invention fulfills this long-standing need and desire in the art.

### SUMMARY OF THE INVENTION

For any of the enveloped RNA viruses studied to date, recognition of a specific RNA packaging signal by the virus's nucleocapsid (N) protein is the first step described in the process of viral RNA packaging. In the murine coronavirus a selective interaction between the viral transmembrane envelope M protein and viral ribonucleoprotein complex composed of N protein and viral RNA containing a short cis-acting RNA element (the packaging signal) determines the selective RNA packaging into virus particles. The present study investigated the mechanism by which mouse hepatitis virus M protein selectively recognizes packaging signal-containing ribonucleoprotein complexes. Expressed M protein specifically interacted with co-expressed non-coronavirus RNA transcripts containing the packaging signal in the absence of N protein. Furthermore, this M protein-packaging signal interaction led to specific packaging of the packaging-signal-containing RNA transcripts into coronavirus-like particles in the absence of N protein.

Thus, mouse hepatitis virus employs a novel mechanism of specific and selective RNA packaging in which the specific interaction between M protein and the packaging signal determines the selectivity and specificity of RNA packaging in the absence of the core or N protein. Furthermore, mouse hepatitis virus M protein is the first viral transmembrane protein that binds to a specific viral RNA element in the absence of any other viral proteins.

These findings not only highlight a novel RNA packaging mechanism for an enveloped virus, where the specific RNA packaging can occur without the core or N protein, but also point to a new, biologically important general model of precise and selective interaction between transmembrane proteins and specific RNA elements.

Thus, it is an object of the present invention to develop a coronavirus-based expression vector system for delivery of RNA and expression of proteins in eukaryotic cells. The findings presented herein indicates that any expressed RNA molecule that contains mouse hepatitis virus packaging signal can be packaged into coronavirus-like particles that contain the coronavirus structural proteins. These coronavirus-like particles would infect mouse hepatitis virus-susceptible cells, resulting in release of the packaged RNA into the cytoplasm and expression of a protein that is encoded by the packaged RNA molecule.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

FIG. 1A shows schematic diagrams of the structures of plasmids PS5A (PS−) and PS5B190(PS+). T7 pr, T7 promoter; T7 ter, T7 terminator; PS, packaging signal.

FIG. 1B shows Northern blot analysis of expressed RNA transcripts from RNA-expressing cells co-infected with SinM and SinN pseudovirions (M protein+N protein).

FIG. 1C shows Northern blot analysis of expressed RNA transcripts from cells infected with either SinM pseudovirion (M protein) or SinLacZ pseudovirion (β-gal protein). Equal volumes of cell lysates were immunoprecipitated with anti-M protein mAb (anti-M) and a control monoclonal antibody (anti-H2K). Intracellular (i.c.) RNAs and co-immunoprecipitated RNAs were analyzed using Northern blot analysis with a probe that binds to the CAT sequence. The arrows indicate expressed RNA transcripts. Each panel shows representative data from triplicate experiments. RNAs extracted from 1×10⁵ cells and 1×10⁶ cells were analyzed on the i.c. RNA lanes, and the anti-M and anti-H2K lanes, respectively. The anti-M and anti-H2K lanes were exposed 8 times longer than the intracellular RNA lanes.

FIG. 2A shows intracellular (i.c.) RNAs extracted from cells expressing PS5B190 (PS+) or PS5A (PS−) RNA transcripts and the MHV-specific proteins. The RNAs were analyzed by Northern blot analysis as described in FIG. 1. The intracellular RNAs extracted from 3×10⁵ cells were applied to each lane.

FIG. 2B shows the release of M protein in coronavirus-like particles. ³⁵S-methionine/cysteine-labelled coronavirus-like particles (VLPs) were purified from culture fluid of the cells expressing PS5B190(PS+) or PS5A (PS−) RNA transcripts and the MHV-specific proteins. A part of the purified VLP lysate was immunoprecipitated with anti-M protein mAb and analyzed by SDS-PAGE. Only the section of the autoradiogram with M protein is shown.

FIG. 2C shows a Northern blot analysis of VLP RNAs. VLP RNA was extracted from purified coronavirus-like particles and analyzed using Northern blot analysis as described above. Coronavirus-like particles released from 1×10⁷ cells were used for analysis of VLP RNAs. FIG. 2C was exposed 8 times longer than FIG. 2A.

FIG. 2D shows intracellular expression levels of M protein and E protein. Cytoplasmic lysates were immunoprecipitated with anti-M protein mAb and anti-E protein peptide-2 antibody and analyzed by SDS-PAGE. Only the sections of the autoradiogram with M protein and E protein are shown. Each panel shows representative data from triplicate experiments.

FIG. 3 shows Northern blot analysis of coronavirus-like particles (VLP)-associated RNAs after RNase A treatment. Partially purified coronavirus-like particles released from cells coexpressing PS5B190 (PS+) RNA transcripts, M protein and E protein were incubated in the presence (RNase+) or absence (RNase−) of RNase A and subsequently purified by ultracentrifugation. Coronavirus-like particles-associated RNAs were extracted from purified coronavirus-like particles. Intracellular (i.c.) RNAs were extracted from the cytoplasmic lysates of the same cells and incubated in the presence (RNAse+) or absence (RNAse−) of RNAse A. Partially purified VLPs released from 2×10⁷ cells and 3 μg of intracellular RNA were used for RNAse A digestion. Northern blot analysis was performed as described in FIG. 1 to examine susceptibility of VLP-associated RNAs and i.c. RNAs to RNase A treatment. Each panel shows representative data from triplicate experiments.

### DETAILED DESCRIPTION OF THE INVENTION

For any of the enveloped RNA viruses studied to date, recognition of a specific RNA packaging signal by the virus's nucleocapsid (N) protein is the first step described in the process of viral RNA packaging. However, mouse hepatitis virus N protein binds to all mouse hepatitis virus mRNAs as well as expressed non-mouse hepatitis virus RNA transcripts in infected cells. This makes it difficult to explain how the formation of N protein-mRNA 1 ribonucleoprotein complex might lead to the specific packaging of RNA into virus particles.

The present study revealed a novel paradigm for viral genome packaging. It is convincingly demonstrated herein that selective interaction of M protein with packaging signal-containing RNA occurs in the absence of N protein. Therefore, recognition of packaging signal-containing RNA by M protein does not require the formation of ribonucleoprotein complex with N protein. Furthermore, it is remarkable that N protein is not necessary for RNA packaging. A specific interaction of a viral envelope protein with a viral RNA element, like the packaging signal, that occurs independently of a nucleocapsid protein with subsequent specific RNA packaging into virus particles (also in the absence of a nucleocapsid protein) has not been described for any RNA virus. Hence, mouse hepatitis virus M protein is the first description of a viral transmembrane protein that binds to a specific viral RNA element in the absence of any other viral structural proteins. These data are consistent with the observation that mouse hepatitis virus M protein co-sediments with mouse hepatitis virus genomic RNA, but not with mouse hepatitis virus N protein, in Renografin density gradient centrifugation of NP-40-solubilized mouse hepatitis virus particles (Sturman et al., 1980).

Currently, it is not clear whether M protein directly interacted with the packaging signal. If M protein directly binds to packaging signal, then mouse hepatitis virus M protein may be the first description of a transmembrane protein that binds to a specific RNA element. M protein-packaging signal binding represents a novel type of macromolecular interaction with a clear biological significance. The nature of binding of M protein to packaging signal thus deserves further study.

Bos et al. (1996) reported the production of infectious mouse hepatitis virus defective interfering (DI) particles in vTF7-3-infected cells that were transfected with five different plasmids expressing the synthetic mouse hepatitis virus DI RNA containing the packaging signal and all four (M, N, S and E) mouse hepatitis virus structural proteins. In that study, culture fluid was collected from expressing cells, and a mixture of the culture fluid and mouse hepatitis virus was used to infect mouse hepatitis virus-susceptible cells. Following overnight incubation, supernatant was collected for subsequent passages. After several undiluted passages of the supernatant, accumulation of DI RNA was demonstrated, implying the packaging of expressed DI RNA into coronavirus like particles in the coexpressing cells. The present data were consistent with their observation that mouse hepatitis virus nonstructural proteins are not necessary for RNA packaging into coronavirus like particles. However, neither the specificity and selectivity of DI RNA packaging nor the role of N protein in DI RNA packaging was examined in the study reported by Bos et al.

Using the Semliki Forest virus (SFV) expression system, others have shown that infectious enveloped particles or vesicles containing the vesicular stomatitis virus envelope G glycoprotein and vector RNA can be produced after expression of the glycoprotein (Rolls et al., 1994). In that system, however, the vector RNA was randomly incorporated into the vesicles. There was no selectivity in RNA packaging as shown herein.

Using the Semliki Forest virus expression system, the random packaging of Semliki Forest virus-derived mRNAs into Semliki Forest virus-encoded murine leukemia virus Gag virus particles was also reported. The Semliki Forest virus-derived mRNAs compensated for the absence of retroviral mRNAs in the virus particles (Muriaux et al., 2001). These mRNAs were packaged despite the lack of any retroviral packaging signal sequences. In a sharp contrast, the present study demonstrated an absolutely specific and selective nucleocapsid-independent packaging of the packaging signal-containing RNA into coronavirus-like particles.

Based on this study and other studies, a model may be proposed to elucidate the mechanism of RNA packaging in mouse hepatitis virus. Since mouse hepatitis virus N protein binds to all mouse hepatitis virus mRNAs in infected cells, probably N protein binds to the intracellular form of genomic RNA, mRNA-1, during nascent mRNA-1 synthesis or as soon as mRNA-1 is synthesized on intracellular membranes. M protein, which accumulates and probably oligomerizes in the intermediate compartment between the ER and the Golgi complex, binds to the packaging signal present in mRNA-1. This binding determines the selective genomic RNA packaging and excludes the packaging of mouse hepatitis virus subgenomic mRNAs lacking the packaging signal. After the binding of M protein to the packaging signal, N protein that is associated with mRNA-1 interacts with the oligomerized M protein. Subsequently, the M protein-mRNA-1 ribonucleoprotein complex undergoes virion morphogenesis in concert with E protein.

These data provoke a question about the biological role of N protein in mouse hepatitis virus. As shown here, N protein appears to be dispensable for mouse hepatitis virus RNA packaging. M-N interaction, however, might compensate for defects in viral envelope formation due to mutation in M protein. N protein may play a crucial role early in infection; for example, one of the functions of N protein may be to deliver the viral ribonucleoprotein complex to the appropriate compartment after virus uncoating to initiate viral replication.

Coronavirus-Based Expression Vector

An object of the present invention is to develop a novel coronavirus-based expression vector system for eukaryotic cells. An important use of this system is delivery of RNA and expression of proteins in eukaryotic cells. Coronavirus-based expression vector system has several advantages over other viral expression systems. Because coronaviruses replicate in the cytoplasm of infected cells without a DNA intermediate, it is unlikely that this virus vector would cause unwanted integration of foreign sequences into host chromosome thereby satisfying many safety concerns. These viruses have the largest RNA genome, which allows for the insertion of large foreign genes. Hence, it is possible to package large RNA molecules into the coronavirus-based expression vector.

Coronaviruses have a broad host range (human, bovine, porcine, canine and feline). The tropism of coronavirus-based expression vector can be engineered by modifying the species of S protein with different receptor recognition ability. Hence, it will be possible to deliver any RNA of interest to specific eukaryotic cells in cell culture as well as in human and animals. Coronaviruses, in general, infect mucosal surfaces. So, the expression of foreign protein (antigen) can be targeted to the enteric and respiratory areas to induce a strong secretory immune response in order to strengthen mucosal defenses. Thus, this system can also be used for the development of novel vaccines.

The finding presented herein indicates that any expressed RNA molecule that contains mouse hepatitis virus packaging signal can be packaged into coronavirus-like particles that contain the coronavirus structural proteins. It is expected that the coronavirus-like particles would infect mouse hepatitis virus-susceptible cells, resulting in the release of the packaged RNA into the cytoplasm and expression of a protein that is encoded by the packaged RNA molecule.

The coronavirus-based expression system can be used as a novel gene delivery system in which non-replicating RNA molecules could be introduced into susceptible cells through infection with coronavirus virus-like particles. Most of the current DNA and RNA virus-based eukaryotic expression vector systems require replication of viral genome for expression of foreign protein of interest. The data shown in the present invention suggest that murine coronavirus-based expression vector could be potentially used to express specific proteins without viral RNA synthesis. Cellular cytopathicity associated with virus replication would not be a potential hazard of this system. The problem of RNA recombination due to RNA replication that leads to generation of wild-type virus would also not be a limitation of this system. A unique aspect of the coronavirus-based expression system described herein is the high specificity in packaging specific RNA molecules into the vector. A single transmembrane viral envelope protein is sufficient to ensure the specificity of RNA species that is packaged into the coronavirus-based vector. This system is quite versatile as it is possible to package both non-replicating and replicating RNA molecules into the vector. In the case of non-replicating RNA, expression of protein will be limited to the cells infected with the vector. In the case of packaging replicating positive-strand RNA virus genome that expresses foreign proteins of interest, cells infected with the vector would support replication of packaged viral RNA genome.

In accordance with the present invention there may be employed conventional molecular biology, microbiology, and recombinant DNA techniques within the skill of the art. Such techniques are explained fully in the literature. See, e.g., Maniatis, Fritsch & Sambrook, “Molecular Cloning: A Laboratory Manual” (1982); “DNA Cloning: A Practical Approach,” Volumes I and II (D. N. Glover ed. 1985); “Oligonucleotide Synthesis” (M. J. Gait ed. 1984); “Nucleic Acid Hybridization” [B. D. Hames & S. J. Higgins eds. (1985)]; “Transcription and Translation” [B. D. Hames & S. J. Higgins eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

The present invention is directed to a coronavirus-based gene delivery and expression vector system. This vector system includes (i) a vector comprising a promoter, a sequence encoding a protein of interest and a packaging signal of a coronavirus, and (ii) a vector or vectors encoding the envelope proteins of a coronavirus. In general, the envelope proteins can be the M protein, E protein, and S protein of a coronavirus. A representative example of a mouse hepatitis virus-based expression vector is described in the present invention.

The present invention also provides a method of expressing a protein of interest in target cells. The method involves first producing coronavirus-based vectors by co-expressing one or more envelope proteins (i.e. the M protein, E protein and S protein) of a coronavirus in cells transfected with a vector comprising a sequence encoding a protein of interest and a packaging signal of a coronavirus. The sequence encoding the protein of interest would be transcribed into RNA molecules which are then packaged into coronavirus particles by the co-expressed coronavirus envelope proteins. These viral particles can then be used to infect target cells, wherein translation of the RNA molecules transferred to the target cells by these viral particles would result in expression of the protein of interest in the target cells. In one embodiment, the RNA molecules are non-replicating RNA molecules so that the expression of the protein of interest is limited to infected target cells. A representative example of a mouse hepatitis virus-based expression vector is described in the present invention.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

Cells and Viruses

DBT (murine astrocytoma) cells were used for DNA transfection and production of VLPs (Hirano et al., 1974), while baby hamster kidney (BHK) cells were used for the preparation of Sindbis pseudovirions. RK13 cells were used for the production and titering of recombinant vaccinia virus vTF7-3 (Fuerst et al., 1986).

### EXAMPLE 2

Preparation of Sindbis Virus Pseudovirions

Sindbis virus vector expressing mouse hepatitis virus S protein (pSinS) was constructed by inserting the entire open reading frame of MHV-2 S protein (Yamada et al., 1997) into Stu I site of a Sindbis virus expression vector, pSinRep5 (Bredenbeek et al., 1993) (Invitrogen, San Diego, Calif.). Sindbis virus pseudovirions, SinM (Maeda et al., 1999), SinE (Maeda et al., 1999), SinN (Narayanan et al., 2000), SinLacZ, and SinS were produced as described (Maeda et al., 1999).

### EXAMPLE 3

DNA Transfection

Sub-confluent monolayers of DBT cells were infected with vTF7-3 at a multiplicity of infection of 5 for 1 hour at 37° C. At one hour postinfection, the cells were transfected with 20 μg of plasmid DNA using a lipofection procedure (Joo et al., 1996), and at 4 hours postinfection the cells were superinfected with Sindbis pseudovirions.

### EXAMPLE 4

Labeling of Proteins, Immunoprecipitation and SDS-PAGE

Infected cells were labeled with 100 μCi of Tran[³⁵S] label/ml of medium for 5 h, from 7 to 12 h post-Sindbis infection. Cell lysates were prepared at 12 h post-Sindbis infection using lysis buffer (1% Triton X-100, 0.5% Na-deoxycholate, 0.1% SDS in phosphate buffered saline [PBS]) (Makino et al., 1991). Intracellular MHV-specific proteins were immunoprecipitated with anti-mouse hepatitis virus N monoclonal antibody (mAb) J3.3, anti-mouse hepatitis virus M monoclonal antibody J1.3 (Fleming et al., 1989), anti-E protein peptide-2 antibody (Yu et al., 1994) and the non-mouse hepatitis virus monoclonal antibody H2K^{k}D^{k} (H2K) as described (Kim et al., 1997). For protein analysis, the immunoprecipitated proteins were incubated at 37° C. for 30 min in sample buffer to prevent M protein aggregation (Sturman et al., 1980) and analyzed using SDS-PAGE. RNAs were extracted from immunoprecipitated samples as described (Narayanan et al., 2000).

### EXAMPLE 5

Purification of Coronavirus-Like Particles

The cell culture media from infected cells was collected at 12 h post-Sindbis pseudovirion infection and briefly centrifuged to remove cell debris. Released radiolabeled coronavirus-like particles were partially purified using ultracentrifugation on a discontinuous sucrose gradient consisting of 50%, 30% and 20% sucrose prepared in NTE buffer (0.1 M NaCl, 0.01 M Tris-HCl [pH 7.5], 0.001 M EDTA) (Maeda et al., 1999). After centrifugation at 26,000 rpm for 16 h at 4° C. in a Beckman SW28 rotor, coronavirus-like particles at the interface of 30% and 50% sucrose were collected. In the case of RNase A treatment, the samples (in sucrose prepared in NTE buffer) were treated with 0.5 ng of RNase A per ml of interface for 30 min at room temperature.

The samples were further purified on a continuous sucrose gradient of 20-60% sucrose at 26,000 rpm for 18 hours at 4° C. The coronavirus-like particles in the fractions corresponding to the reported density of coronavirus-like particles (1.14 to 1.16 g/cm³) (Maeda et al., 1999; Vennema et al., 1996) were collected and pelleted using ultracentrifugation in a Beckman SW28 rotor at 26,000 rpm for 3 h at 4° C. The pellets were suspended in the lysis buffer.

### EXAMPLE 6

Analysis of Coronavirus-Like Particles RNA and Intracellular RNA

Purified coronavirus-like particles (VLPs) were suspended in the lysis buffer and RNA was extracted from VLP lysates using established methods (Makino et al., 1988). The intracellular RNA was extracted from cytoplasmic lysates as described (Makino et al., 1984). After DNase treatment (Woo et al., 1997), RNAs were denatured and separated on a 1% agarose gel containing formaldehyde (Makino et al., 1991). After electrophoresis, Northern blot analysis was performed using digoxigenin-labeled random-primed probe (Boehringer) specific to the chloramphenicol acetyltransferase (CAT) gene (Narayanan et al., 2000; Woo et al., 1997). The RNAs were visualized using DIG luminescent detection kit (Boehringer).

### EXAMPLE 7

Envelope M Protein Selectively Interacts With Packaging Signal-Containing Non-Mouse Hepatitis Virus RNA Transcript In The Absence of N Protein

The specific and selective interaction between M protein and packaging signal-containing ribonucleoprotein complexes drives the specific packaging of packaging signal-containing RNAs into mouse hepatitis virus particles (Narayanan and Makino, 2001). In all known enveloped RNA viruses studied thus far, N protein or capsid protein plays an essential role in viral RNA packaging. However, the role(s) of N protein in mouse hepatitis virus RNA packaging is unknown.

How M protein selectively recognizes packaging signal-containing ribonucleoprotein complexes was examined in co-expression experiments. These experiments test packaging of non-mouse hepatitis virus RNA with inserted packaging signal in the presence of combinations of various expressed mouse hepatitis virus proteins. It is of particular interest to determine whether N protein is essential for the selective interaction between M protein and packaging signal-containing ribonucleoprotein complexes.

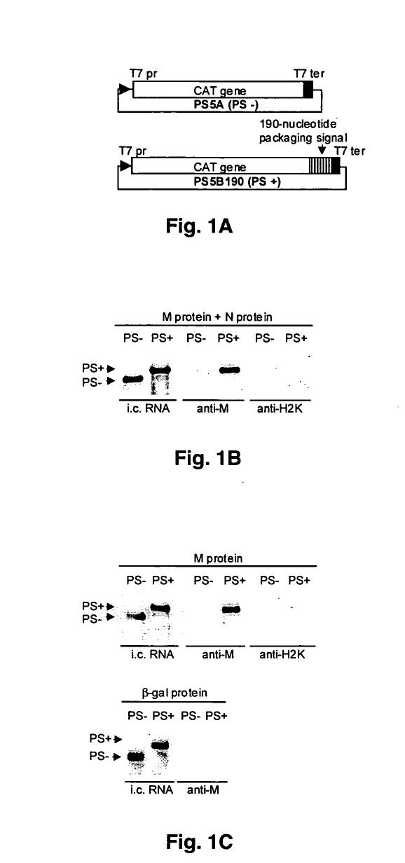

DBT cells were infected with a recombinant vaccinia virus, vTF7-3, which encodes the T7 RNA polymerase (Fuerst et al., 1986). One hour later, the cells were independently transfected with either plasmid PS5A that contains the entire CAT gene under the dual controls of the T7 promoter and the T7 terminator, or with plasmid PS5B190 that carries the mouse hepatitis virus 190-nt packaging signal positioned downstream of the CAT gene (FIG. 1A). RNA transcripts are expressed from transfected PS5A and PS5B190 in vTF7-3-infected cells (Narayanan and Makino, 2001; Woo et al., 1997). At 4 hours post vTF7-3 infection, which was 3 hours post plasmid transfection, cultures from both plasmid transfections were superinfected with one or combinations of three Sindbis virus expression vectors: SinM pseudovirion (expressing mouse hepatitis virus M protein); SinN pseudovirion (expressing mouse hepatitis virus N protein) or SinLacZ pseudovirion (encoding the β-galactosidase protein) (Maeda et al., 1999; Narayanan et al., 2000). Cell extracts were prepared at 12 h post-Sindbis pseudovirion infection and used for co-immunoprecipitation analysis with anti-M monoclonal antibody or control anti-H2K monoclonal antibody. RNA was extracted from the immunoprecipitated samples and treated with DNase (Narayanan and Makino, 2001; Woo et al., 1997).

Northern blot analysis with a CAT sequence-specific probe showed that PS5A and PS5B190 RNA transcripts were expressed at similar levels (FIG. 1). Strikingly, anti-M monoclonal antibody co-immunoprecipitated PS5B190 RNA transcript from cells coexpressing PS5B190 RNA transcript and the M protein as well as from cells co-expressing M protein and N protein with the same transcript (FIGS. 1B, 1C). Anti-M monoclonal antibody did not co-precipitate PS5A transcripts from co-expressing cells, nor did it co-precipitate PS5B190 transcripts from cells co-expressing β-galactosidase. Anti-H2K monoclonal antibody did not coimmunoprecipitate either PS5B190 or PS5A transcripts, establishing that the co-immunoprecipitation using anti-M monoclonal antibody was specific. Consistent with a previous study (Narayanan et al., 2000), SDS-PAGE analysis showed that only M monoclonal antibody and not H2K mAb immunoprecipitated M protein (data not shown).

These data demonstrated that co-expressed M protein bound to expressed packaging signal-containing RNA transcripts in the absence of other mouse hepatitis virus functions, including N protein. Furthermore, these data strongly suggested that the packaging signal was a signal for binding the envelope transmembrane protein M.

### EXAMPLE 8

Packaging Signal-Containing RNAs Are Selectively Packaged Into Coronavirus-Like Particles In The Absence of N Protein

The finding that M protein bound to packaging signal-containing RNA transcripts in the absence of N protein led to the investigation of whether the packaging signal-containing RNA transcripts could be packaged into coronavirus-like particles (VLPs) in the absence of N protein. Expression of two coronavirus envelope proteins, M and E, resulted in the production of coronavirus-like particles, which were indistinguishable from authentic coronavirions in size and shape (Bos et al., 1996; Vennema et al., 1996). S protein is non-essential for coronavirus assembly.

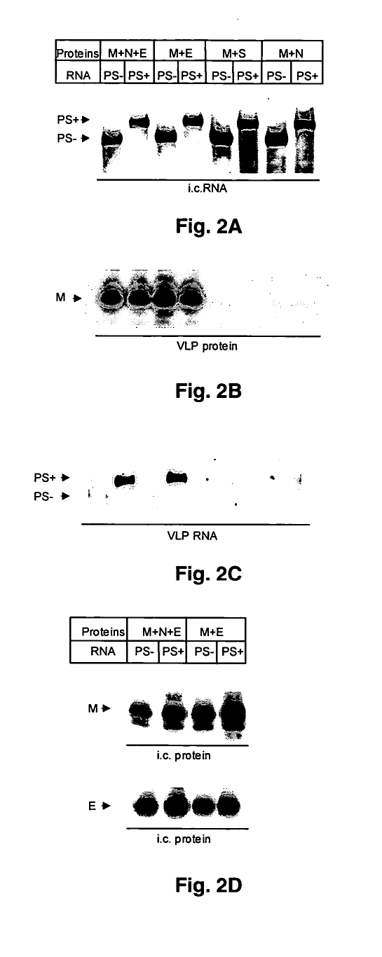

vTF7-3-infected DBT cells were independently transfected with the PS5B190 plasmid or the PS5A plasmid. The cells were superinfected with a mixture of Sindbis pseudovirions. PS5A transcript served as a negative control for testing the specificity of RNA packaging. Because co-expression of M and E protein is required for coronavirus-like particle production (Vennema et al., 1996), omission of E protein expression served as a negative control for the coronavirus-like particle production. Cells were radiolabeled and culture fluids and cell extracts from co-expressing cells were collected at 12 h post Sindbis pseudovirion infection. Both the PS5B190 and PS5A RNA transcripts were expressed similarly in all the samples (FIG. 2A). The released coronavirus-like particles (VLPs) were purified using sucrose gradient centrifugation, and the fractions corresponding to VLP density were collected. The corresponding fractions in the negative control samples were also collected. Coronavirus-like particle production was measured through detection of M protein in the sucrose fractions. Similar amount of coronavirus-like particles were produced from the cells coexpressing M and E proteins or M, N and E proteins.

As expected, coronavirus-like particles were not produced from cells co-expressing M and S proteins or co-expressing M and N proteins (FIG. 2B). A similar amount of PS5B190 transcript was easily detected in the released coronavirus-like particles from cells co-expressing M and E proteins and the PS5B190 RNA transcripts, as well as from cells additionally co-expressing N protein (FIG. 2C).

In contrast, only a very low level of PS5A transcript was detected in the released coronavirus-like particles (FIG. 2C). Also, only trace amounts of expressed RNA transcripts were detected in the supernatant from cells co-expressing M and N proteins or M and S proteins (FIG. 2C). Analysis of the intracellular proteins showed that both M and E proteins accumulated to similar levels in the expressing cells (FIG. 2D). N and S proteins also accumulated to similar levels in the expressing cells (data not shown). These data demonstrated that co-expression of M, N and E proteins and the RNA containing the packaging signal resulted in the production of coronavirus-like particles containing the RNA transcripts. Most importantly, this data convincingly demonstrated that co-expression of M and E proteins and RNA containing the packaging signal resulted in the production of coronavirus-like particles containing the RNA transcripts. Surprisingly, N protein was dispensable for RNA packaging.

To further confirm that the PS5B190 RNA transcripts were indeed packaged within the coronavirus-like particles, the samples containing the released coronavirus-like particles were treated with RNase A. If the RNA transcripts are present within the coronavirus-like particles, then the RNAs should be inaccessible to RNase A and hence resistant to RNase A treatment. Partially purified coronavirus-like particles released from cells expressing PS5B190 RNA transcripts and M protein and E protein were incubated in the presence of RNase A. Subsequently the coronavirus-like particles were purified by ultracentrifugation and the RNAs were extracted from the purified coronavirus-like particles. As a control, the intracellular RNAs extracted from the same cells were also subjected to the same RNase A treatment under the same buffer conditions.

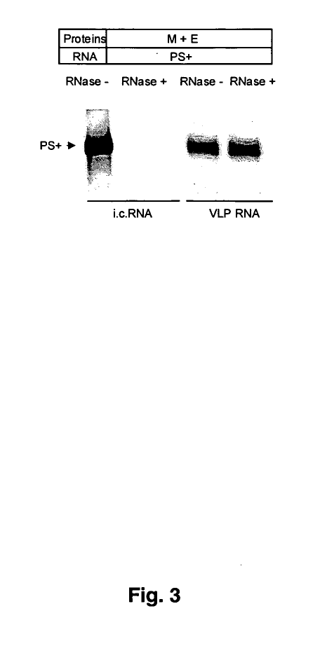

Northern blot analysis revealed that the intracellular RNAs extracted from the same cells were completely degraded by the RNase A treatment (FIG. 3), demonstrating that the experimental condition for RNase treatment was appropriate. In contrast, no degradation of PS5B190 RNA transcripts occurred after RNase A treatment of the coronavirus-like particles (FIG. 3), demonstrating that PS5B190 RNA transcripts were indeed selectively packaged into coronavirus-like particles.

### EXAMPLE 9

Infectivity of Coronavirus-Like Particles (VLPs)

Coronavirus-based expression vector should be able to deliver foreign genes to virus-susceptible cells. To confirm that coronavirus-like particles can be used to express heterologous RNA and protein in target cells, coronavirus-like particles containing M, E, S proteins and the packaged RNA transcript will be produced from cells co-expressing MHV structural proteins (M, E and S) and the RNA transcripts containing the PS. The RNA transcripts will be expressed using the vaccinia virus T7 expression system, while Sindbis pseudovirions will be used for the expression of the structural proteins. These coronavirus-like particles containing the packaged RNA transcripts will be used to infect coronavirus-susceptible cells, to monitor the delivery and expression of foreign gene of interest. The packaged RNA transcripts can either be self-replicating positive-strand RNA virus genome or non-replicating RNA molecules. The RNA transcript can be engineered to express a reporter gene like green fluorescent protein (GFP), β-galactosidase or luciferase to monitor the expression of protein in target cells. In the case of non-replicating RNA molecules, the translation of RNA transcript, introduced into target cells by VLPs, will result in the expression of reporter protein, which can be easily analyzed. In the case of replicating RNA molecules, amplification of RNA transcripts in target cells can be assayed by Northern blot.

### EXAMPLE 10

Alternative Strategy to Produce Coronavirus-Based Expression Vector

Coronavirus-like particles containing RNA transcript are produced from cells co-expressing MHV structural proteins and RNA transcript containing the PS. Instead of using vaccinia virus T7 expression system to express the RNA transcripts and the Sindbis expression system to express MHV structural proteins, an alternative method can be used to express the RNA transcripts and the MHV structural proteins. The cDNA, encoding the foreign gene of interest and the MHV PS, can be cloned downstream of the cytomegalovirus promoter (CMV). Similarly, all the MHV structural protein genes (M, E, and S proteins) can also be cloned downstream of the CMV promoter. Co-transfection of the DNA plasmid encoding the foreign gene of interest and the plasmids encoding MHV structural proteins (M, E, S) into cells will result in the expression of RNA transcripts under the control of the CMV promoter using the cellular RNA polymerase II. Translation of the RNA transcripts will result in the expression of MHV structural proteins. Co-expression of the RNA transcript, encoding the foreign gene and MHV PS, along with MHV structural proteins will result in the production of coronavirus-like particles containing the packaged RNA transcript. These coronavirus-like particles can be used as expression vectors to deliver the gene of interest to target cells.

### EXAMPLE 11

Coronavirus-Alphavirus Hybrid Expression Vectors

One of the advantages of coronavirus-based expression vector is that large RNA molecules can be packaged into coronavirus-like particles. Another feature of this system is that the packaging signal of MHV will drive the specific packaging of any RNA molecule into coronavirus-like particles. Coronavirus-based system can be adapted to package recombinant self-replicating replicon RNAs from other viruses into coronavirus-like particles. An example of such a hybrid vector is provided below.

A self-replicating replicon based on the alphavirus, Venezuelan equine encephalitis virus (VEE), has been used as recombinant alphavirus expression vector to express heterologous proteins to high levels in susceptible cells. The self-replicating, Venezuelan equine encephalitis virus replicon was generated by replacing the genes for the alphavirus structural protein with that of a reporter protein (Xiong C et al, 1989, Science, 243, 1188-1191). The Venezuelan equine encephalitis virus replicon can be engineered to contain the heterologous gene of interest and MHV PS, which will drive the specific packaging of replicon RNA transcripts into coronavirus-like particles. The Venezuelan equine encephalitis virus replicon RNA can be packaged into coronavirus-like particles by co-expression of MHV structural proteins (M, E and S) and Venezuelan equine encephalitis virus replicon containing MHV PS to generate hybrid expression vectors. One of the advantages of these hybrid vectors is that the problem of generation of wild-type Venezuelan equine encephalitis virus viruses, as a result of RNA recombination, can be eliminated because MHV structural proteins, instead of Venezuelan equine encephalitis virus structural proteins, are used to package Venezuelan equine encephalitis virus replicons into coronavirus-like particles. Another advantage of such hybrid vectors is that the expression of foreign proteins can be restricted to specific target cells, determined by coronavirus S protein.

Coronavirus-like particles can be engineered to express novel proteins in their envelopes. For example, a chimeric expression vector encoding a fusion protein between HIV envelope glycoprotein and MHV M protein can be used to incorporate HIV envelope glycoprotein into coronavirus-like particles. Co-expression of this fusion protein and the MHV structural proteins (M and E) along with Venezuelan equine encephalitis virus replicon RNA, encoding specific gene of interest and MHV PS, will result in the production of coronavirus-like particles containing the packaged Venezuelan equine encephalitis virus replicon RNA. This hybrid coronavirus-based vector will express the chimeric HIV envelope protein on its envelope. The tropism of such expression vectors will be determined by HIV envelope glycoprotein. These virus-like particles will selectively enter HIV-susceptible cells and can be used as targeted gene delivery vectors. Similar targeted gene delivery vectors can be generated by expressing other viral envelope proteins (like vesicular stomatitis virus G protein) as chimeric proteins on the surface of coronavirus-like particles. These gene delivery vectors have the potential to deliver RNA molecules encoding specific toxins to destroy virus-infected cells or even cancer cells.

The following references were cited herein:

Armstrong et al., 1984. Nature 308:751-752.

Baric et al., 1988. J Virol. 62:4280-4287.

Bos et al., 1996. Virology 218:52-60.

Bredenbeek et al., 1993. J Virol. 67:6439-6446.

Escors et al., 2001. J Virol. 75:1312-1324.

Fischer et al., 1998. J Virol. 72:7885-7894.

Fleming et al., 1989. Virology 168:162-167.

Fosmire et al., 1992. J Virol. 66:3522-3530.

Fuerst et al., 1986. Proc Natl Acad Sci USA. 83:8122-8126.

Hirano et al., 1974. Arch Gesamte Virusforsch. 44:298-302.

Joo et al., 1996. J Virol. 70:5769-5776.

Kim et al., 1997. Assembled coronavirus from complementation of two defective interfering RNAs. J Virol. 71:3922-3931.

Lai et al., 1984. Proc Natl Acad Sci USA. 81:3626-3630.

Lai and Cavanagh, 1997. Adv. Virus Res. 48:1-100.

Lai and Stohlman, 1978. J Virol. 26:236-242.

Maeda et al., 1999. Virology 263:265-272.

Makino et al., 1991. J Virol. 65:6031-6041.

Makino et al., 1988. Virology 163:104-111.

Makino et al., 1984. Virology 139:138-151.

Muriaux et al., 2001. Proc Natl Acad Sci USA. 98:5246-5251.

Narayanan et al., 2000. J Virol. 74:8127-8134.

Narayanan and Makino, 2001. J Virol. 75:9059-9067.

Rolls et al., 1994. Cell 79:497-506.

Spaan et al., 1983. EMBO J. 2:1839-1844.

Sturman et al., 1980. J Virol. 33:449-462.

van der Most et al., 1991. J Virol. 65:3219-3226.

Vennema et al., 1996. EMBO J. 15:2020-2028.

Woo et al., 1997. J Virol. 71:824-827.

Yamada et al., 1997. Virology 227:215-219.

Yu et al., 1994. Virology 202:1018-1023.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

What is claimed is:

1. A coronavirus-based gene delivery and expression vector system comprising:
a vector comprising a promoter, a sequence encoding a protein of interest and a packaging signal of a coronavirus; and
a vector or vectors encoding the envelope protein(s) of a coronavirus.

2. The expression vector system of claim 1, wherein said vector encoding said envelope protein is a plasmid vector or a viral vector.

3. The expression vector system of claim 1, wherein said envelope protein is selected from the group consisting of the M protein, E protein, and S protein of a coronavirus.

4. The expression vector system of claim 1, wherein said coronavirus is mouse hepatitis virus.

5. The expression vector system of claim 1, wherein transcription of said vector encoding said protein of interest results in a non-replicating or replicating RNA molecule encoding said protein of interest.

6. A method of expressing a protein of interest in target cells, said method comprises the steps of:
transfecting a first set of cells with a vector, said vector comprises a promoter, a sequence encoding a protein of interest and a packaging signal of a coronavirus;
transcribing said sequence encoding said protein of interest into RNA molecules;
transfecting or infecting said first cells with a vector or vectors encoding one or more of the envelope proteins of a coronavirus;
packaging said RNA molecules and said envelope protein(s) into viral particles;
collecting said viral particles comprising said RNA molecules encoding said protein of interest; and
infecting target cells with said viral particles, wherein translation of said RNA molecules transferred to said target cells by said viral particles results in expression of said protein of interest in said target cells.

7. The method of claim 6, wherein said envelope protein is selected from the group consisting of the M protein, E protein, and S protein of a coronavirus.

8. The method of claim 6, wherein said coronavirus is mouse hepatitis virus.

9. The method of claim 6, wherein said RNA molecules are replicating RNA molecules.

10. The method of claim 6, wherein said RNA molecules are non-replicating RNA molecules so that the expression of said protein of interest is limited to infected target cells.
